(19) United States
(12) Patent Application Publication
(10) Pub. No.: US 2017/0319081 A1
(43) Pub. Date: Nov. 9, 2017

(54) ELECTRONIC DEVICE AND CONTROL METHOD

(71) Applicants: KYUSHU UNIVERSITY, NATIONAL UNIVERSITY CORPORATION, Fukuoka (JP); KYOCERA Corporation, Kyoto (JP)

(72) Inventors: Renshi SAWADA, Fukuoka-shi, Fukuoka (JP); Hirofumi NOGAMI, Fukuoka-shi, Fukuoka (JP); Terukazu AKIYAMA, Hachioji-shi, Tokyo (JP); Ryo UENO, Uji-shi, Kyoto (JP); Kazuhiro UMEDA, Mita-shi, Hyogo (JP); Takuya FUJIWARA, Yokohama-shi, Kanagawa (JP); Tomoaki TAKASHIMA, Sagamihara-shi, Kanagawa (JP)

(73) Assignees: KYUSHU UNIVERSITY, NATIONAL UNIVERSITY CORPORATION, Fukuoka (JP); KYOCERA Corporation, Kyoto (JP)

(21) Appl. No.: 15/520,060

(22) PCT Filed: Oct. 20, 2015
(86) PCT No.: PCT/JP2015/079619

§ 371 Date: Apr. 18, 2017

(30) Foreign Application Priority Data
Oct. 21, 2014 (JP) 2014-214667

(51) Int. Cl. A61B 5/02; A61B 5/026; A61B 5/0285; A61B 5/00

(52) U.S. Cl. CPC A61B 5/02035; A61B 5/7278; A61B 5/0261; A61B 5/0285

(57) ABSTRACT

An electronic device according to one aspect includes a blood flow data acquisition unit configured to acquire information related to blood flowing inside a living body as blood flow data based on Doppler shift, a power spectrum calculator configured to calculate a power spectrum of the blood flow data based on the blood flow data, and an outline index calculator configured to calculate an outline index from the power spectrum. An electronic device according to one aspect further includes an estimator configured to estimate viscosity of the blood based on the outline index, and the estimator is configured to display an estimation result of the viscosity of the blood on a display.

### FIELD

The present application relates to an electronic device and a control method.

### BACKGROUND

There is a conventional technology for analyzing fluidity of blood. For example, there is a technology in which a Doppler shift signal corresponding to a blood flow is acquired by capturing a reflected wave of a wave motion with which a surface of a skin is irradiated, and fluidity of blood is analyzed based on a velocity of the blood flow calculated from the Doppler shift signal.

### SUMMARY

An electronic device according to one aspect includes a blood flow data acquisition unit configured to acquire information related to blood flowing inside a living body as blood flow data based on Doppler shift, a power spectrum calculator configured to calculate a power spectrum of the blood flow data based on the blood flow data, and an outline index calculator configured to calculate an outline index from the power spectrum.

A control method according to one aspect executed by an electronic device includes the steps of acquiring information related to blood flowing inside a living body as blood flow data based on Doppler shift, calculating a power spectrum of the blood flow data based on the blood flow data, and calculating each of slopes of the power spectrum corresponding to a plurality of different frequencies, respectively, as an outline index.

### BRIEF DESCRIPTION OF DRAWINGS

FIG. 1 is a diagram illustrating an example of an external configuration of an electronic device according to some embodiments.

FIG. 2 is a diagram illustrating an example of an external configuration of the electronic device according to embodiments.

FIG. 3 is a diagram illustrating an example of a measurement state of blood flow data.

FIG. 4 is a block diagram illustrating an example of a function configuration of the electronic device according to embodiments.

FIG. 5 is a diagram illustrating an example of reference data related to pressure determination at the time of measuring blood flow data.

FIG. 6 is a diagram illustrating an example of a procedure for calculating a power spectrum.

FIG. 7 is a diagram illustrating an example of a power spectrum of blood flow data having different viscosities.

FIG. 8 is a diagram for description of a procedure for calculating an outline index.

FIG. 9 is a diagram for description of a procedure for calculating an outline index.

FIG. 10 is a diagram illustrating an example of a waveform representing a relation between a blood flow rate and time.

FIG. 11 is a diagram illustrating a display example of an evaluation result of blood viscosity.

FIG. 12 is a flowchart illustrating an overall flow of a process by the electronic device according to some embodiments.

FIG. 13 is a flowchart illustrating a flow of a process of determining blood viscosity by the electronic device according to some embodiments.

FIG. 14 is a diagram illustrating an example of outline indices corresponding to different test subjects.

FIG. 15 is a diagram illustrating an example of blood flow rates corresponding to different test subjects.

### DESCRIPTION OF EMBODIMENTS

The above-described technology is used to analyze fluid of blood, but not used to analyze blood viscosity. For this reason, blood viscosity may not be analyzed non-invasively and in a short time. In this regard, there is a need to provide an electronic device and a control method capable of analyzing blood viscosity non-invasively and in a short time. Embodiments for implementing the invention will be described in detail with reference to drawings.

### Embodiments

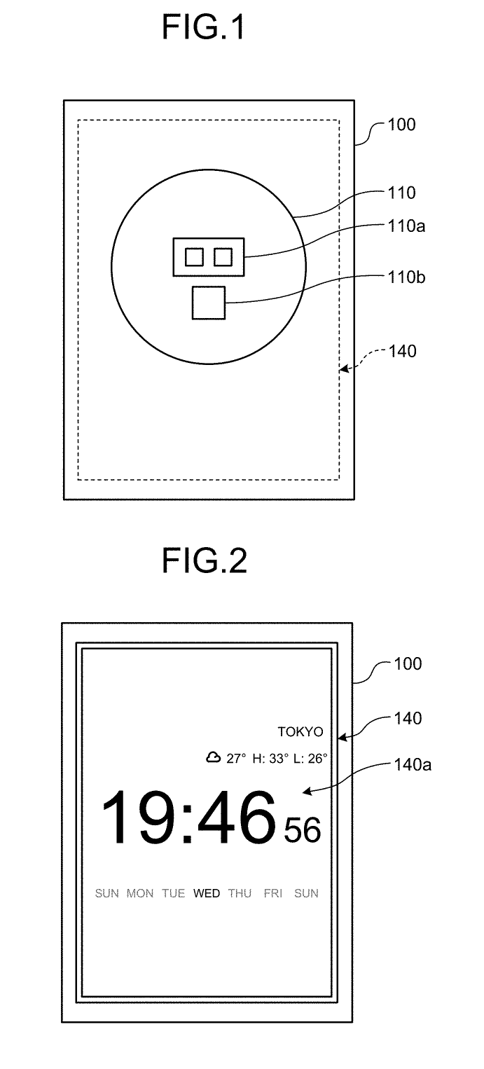

FIG. 1 and FIG. 2 are diagrams illustrating examples of an external configuration of an electronic device 100 according to some embodiments. FIG. 1 illustrates a first surface included in the electronic device 100, and FIG. 2 illustrates a second surface included in the electronic device 100.

In the example illustrated in FIG. 1, a measurement unit 110 is provided on the first surface included in the electronic device 100. For example, cover glass is installed on a surface of the measurement unit 110. The measurement unit 110 includes a blood flow sensor 110a and a pressure sensor 110b.

In the example illustrated in FIG. 2, a display 140 is provided on the second surface included in the electronic device 100. For example, the display 140 may display a standby screen 140a while measurement of blood flow data is performed. The display 140 is an example of a display.

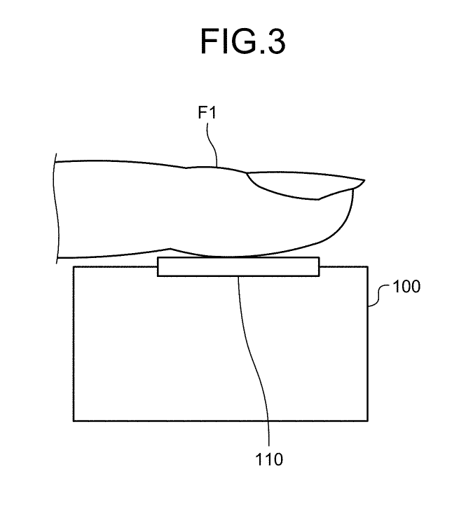

FIG. 3 is a diagram illustrating an example of a measurement state of blood flow data. As illustrated in FIG. 3, in response to a finger F1 of a user being disposed on the surface of the measurement unit 110, the electronic device 100 performs measurement of a pressure on the measurement unit 110, and starts to measure blood flow data when the pressure satisfies a predetermined condition. Referring to a measurement time of the blood flow data measured by the electronic device 100, while a minimum of a measurement time corresponding to a time of one pulse beat needs to be ensured, a possibility that noise due to movement of a human will be mixed with measurement data increases as the measurement time increases. Thus, a suitable measurement time is about two seconds to four seconds.

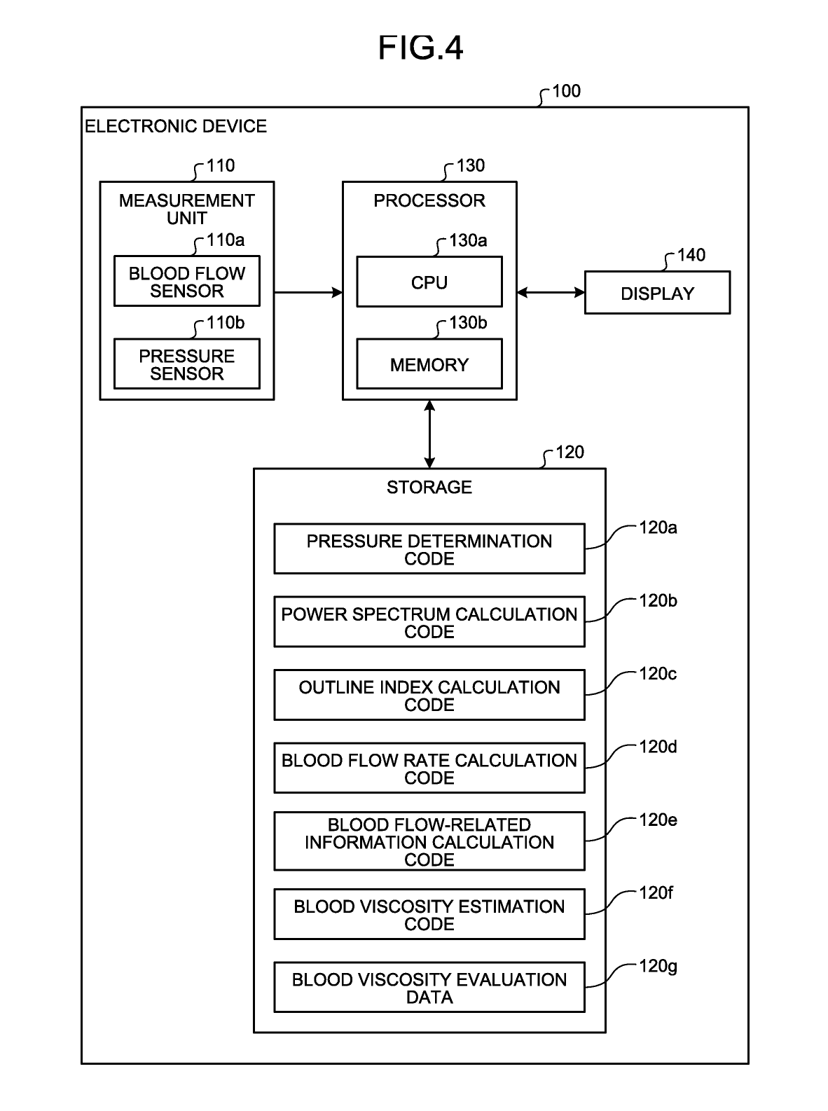

FIG. 4 is a block diagram illustrating an example of a function configuration of the electronic device 100 according to embodiments. As illustrated in FIG. 4, the electronic device 100 includes the measurement unit 110, storage 120, a processor 130, and the display 140.

The measurement unit 110 includes the blood flow sensor 110a and the pressure sensor 110b.

The blood flow sensor 110a acquires information related to blood flowing inside a living body as blood flow data based on Doppler shift. The blood flow sensor 110a irradiates a portion around blood flowing through a blood vessel with laser light from a light emitter. The blood flow sensor 110a receives scattered light from a material inside the body including scattered light from the blood using the same light emitter. The blood flow sensor 110a calculates data related to a velocity of the blood based on a difference in wavelength of scattered light (Doppler shift) from the blood, and acquires the calculated data as blood flow data. The laser light emitted from the light emitter may correspond to light having a wavelength of 1.31 micrometer which has a high skin permeation rate, and a small amount of which is absorbed by hemoglobin. The light emitter may correspond to a distributed feedback laser that oscillates in a single longitudinal mode. The blood flow sensor 110a may correspond to a laser irradiation-type sensor or a sonic irradiation-type sensor. The blood flow sensor 110a is an example of a blood flow data acquisition unit.

The pressure sensor 110b measures a pressure on the measurement unit 110 at the time of measuring the blood flow data. The pressure sensor 110b measures distortion of the cover glass provided on the surface of the measurement unit 110, and converts the measured distortion into a pressure. In place of mounting the pressure sensor 110b, the cover glass may be allowed to function as a pressure sensor by configuring the cover glass provided on the surface of the measurement unit 110 as a translucent piezoelectric element.

The storage 120 stores a code and data. The storage 120 stores a code and data necessary for various processes executed by the processor 130. The storage 120 may include an arbitrary non-transitory storage medium such as a semiconductor storage medium and a magnetic storage medium. The storage 120 may include a plurality of types of storage media. The storage 120 may include a combination of a portable storage medium such as a memory card, an optical disc, and a magneto-optical disc, and a reader for the storage medium. The storage 120 may include a storage device used as a temporary storage area such as a random access memory (RAM).

In the example illustrated in FIG. 4, the storage 120 stores a pressure determination code 120a, a power spectrum calculation code 120b, an outline index calculation code 120c, a blood flow rate calculation code 120d, a blood flow-related information calculation 120e, a blood viscosity estimation code 120f, and blood viscosity evaluation data 120g. The power spectrum calculation code 120b is an example of a power spectrum calculator. The outline index calculation code 120c is an example of an outline index calculator. The blood flow rate calculation code 120d is an example of a blood flow rate calculator. The blood flow-related information calculation code 120e is an example of a blood flow-related information calculator. The blood viscosity estimation code 120f is an example of an estimator.

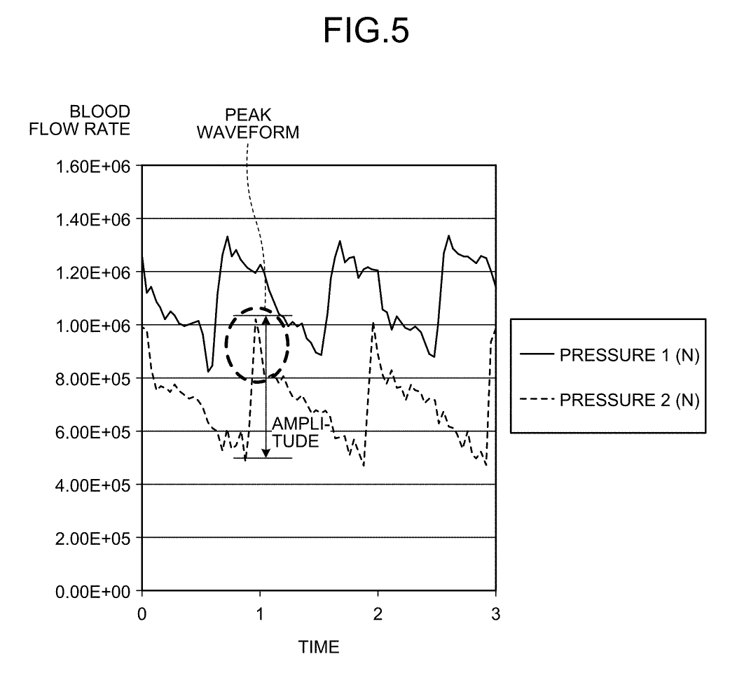

The pressure determination code 120a provides a function for executing a process of determining a pressure on the measurement unit 110 at the time of measuring blood flow data. For example, when a contact to the measurement unit 110 is detected, the pressure determination code 120a determines whether a pressure on the measurement unit 110 is stable around a predetermined numerical value. FIG. 5 is a diagram illustrating an example of reference data related to pressure determination at the time of measuring blood flow data. FIG. 5 illustrates a waveform indicating a temporal change of a blood flow rate when the pressure is 1 Newton (N), and a waveform indicating a temporal change of a blood flow rate when the pressure is 2 N. As illustrated in FIG. 5, a peak waveform appearing for each one pulse beat is clearer in the waveform indicating the temporal change of the blood flow rate when the pressure is 2 N than in the waveform when the pressure is 1 N. In this regard, the pressure determination code 120a determines whether the pressure on the measurement unit 110 is stable around 2 N. For example, when the pressure on the measurement unit 110 is in a range of 2 N±0.1 N during a predetermined determination time, the pressure determination code 120a determines that the pressure is stable around 2 N.

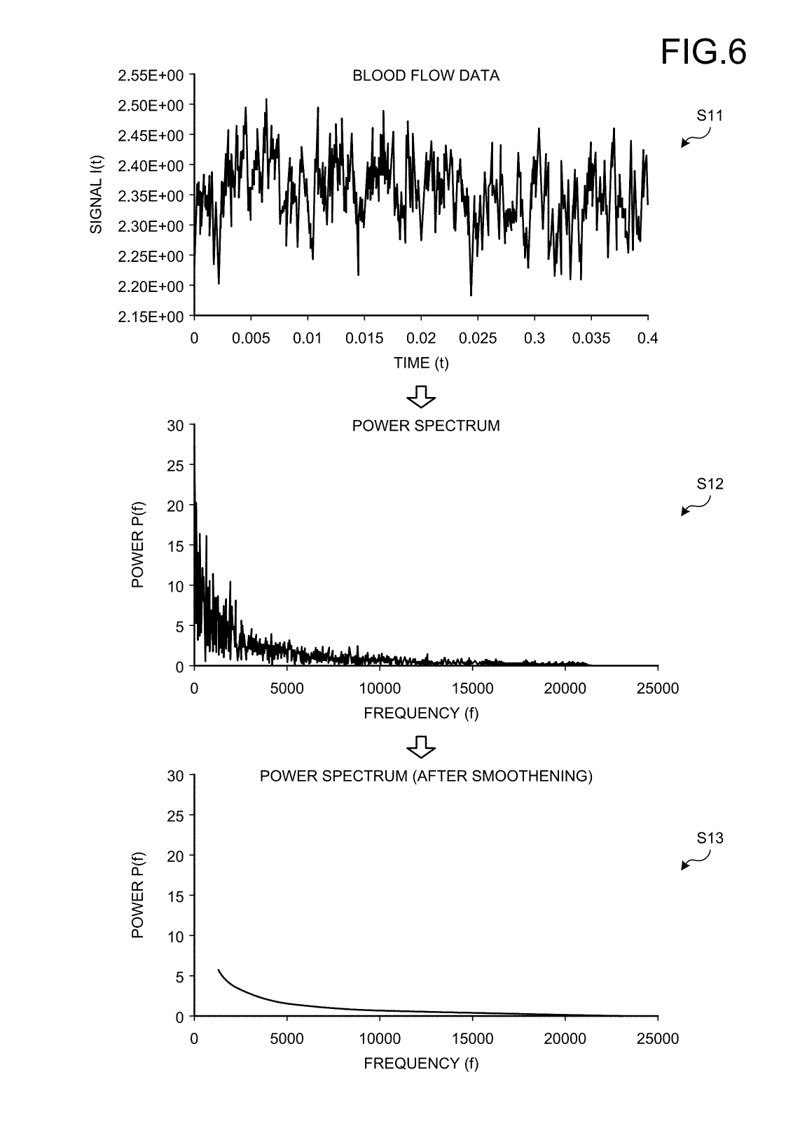

The power spectrum calculation code 120b provides a function for executing a process of calculating a power spectrum of the blood flow data based on the blood flow data acquired by the blood flow sensor 110a. FIG. 6 is a diagram illustrating an example of a procedure for calculating a power spectrum. As illustrated in FIG. 6, the power spectrum calculation code 120b samples blood flow data during 0.04 second from the blood flow data acquired by the blood flow sensor 110a (Step S11). Subsequently, the power spectrum calculation code 120b calculates a power spectrum of the blood flow data by Fourier-transforming the sampled blood flow data (Step S12). Subsequently, the power spectrum calculation code 120b smoothens the calculated power spectrum (Step S13). The power spectrum calculation code 120b calculates respective power spectrums with respect to all blood flow data sampled during a predetermined measurement time (for example, three seconds) of the blood flow data. Smoothening of the power spectrum is performed to clarify an outline of the power spectrum, and may not be performed.

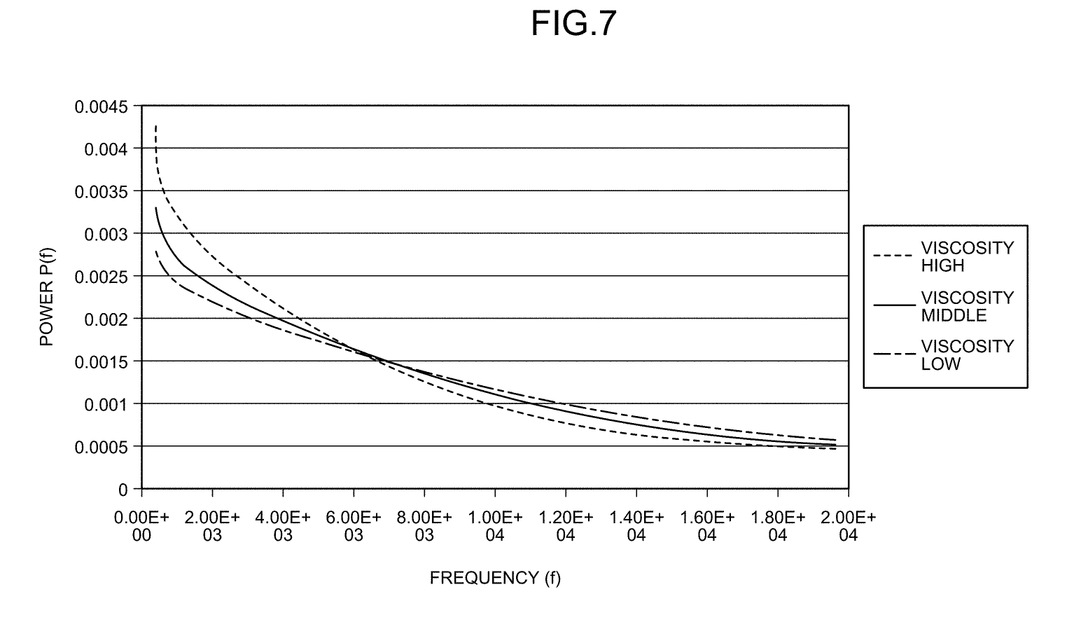

FIG. 7 is a diagram illustrating an example of a power spectrum of blood flow data having different viscosities. As illustrated in FIG. 7, power in a low frequency region is prone to increase as blood viscosity acquired as blood flow data increases, and power in a high frequency region is prone to decrease as the viscosity decreases. The present embodiment focuses on a tendency illustrated in FIG. 7, and attempts to calculate an outline index from a power spectrum using the outline index calculation code 120c described below.

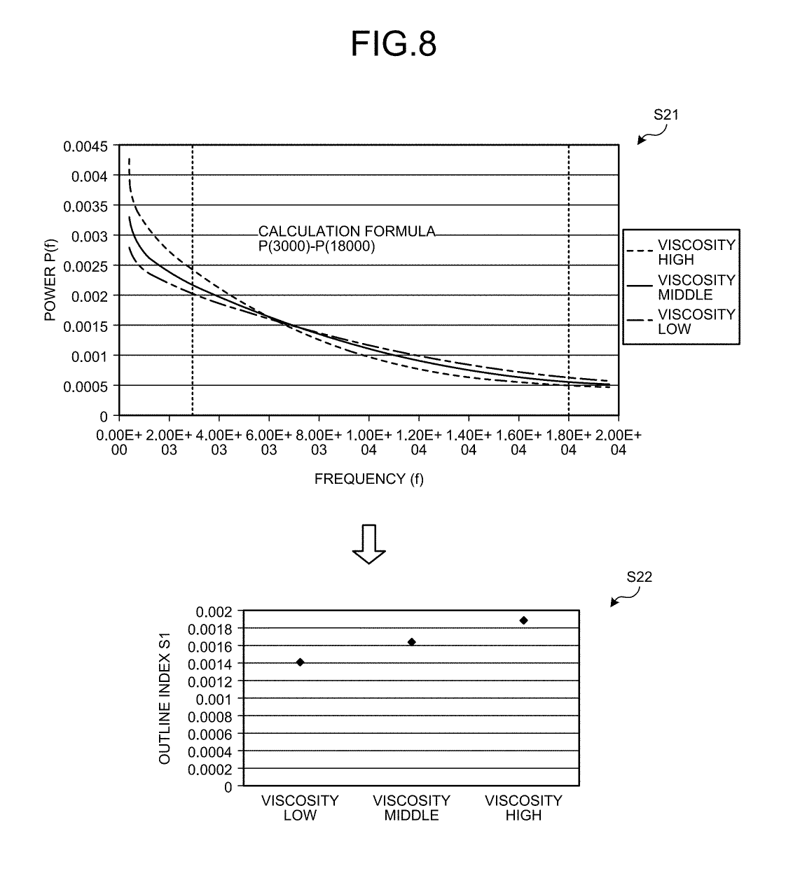

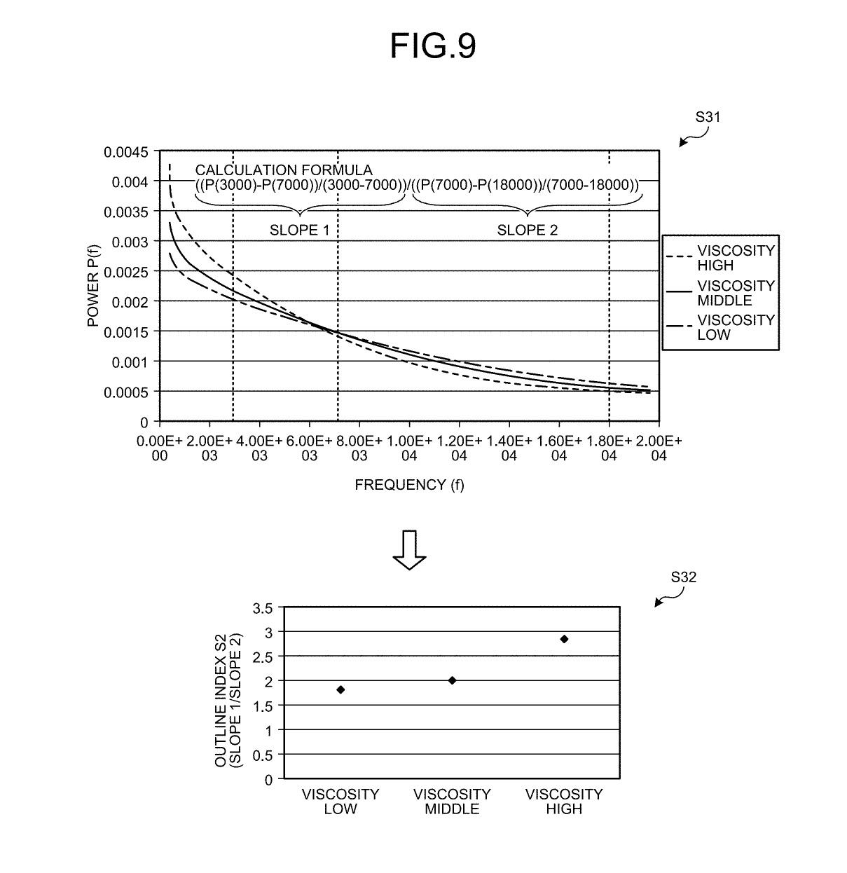

The outline index calculation code 120c provides a function for executing a process of calculating an outline index from a power spectrum. The outline index represents a characteristic of a waveform of a power spectrum using a numerical value. For example, the outline index corresponds to slopes of tangents at a plurality of different frequencies, a ratio of the slopes, a difference between powers at a plurality of different frequencies, and a ratio of the powers. The outline index may be calculated based on powers at three or more different frequencies to represent nonlinearity of the waveform of the power spectrum. Hereinafter, a description will be given of a procedure for calculating an outline index using FIG. 8 and FIG. 9. FIG. 8 and FIG. 9 are diagrams for description of the procedure for calculating the outline index.

Using FIG. 8, a description will be given of a procedure in which the outline index calculation code 120c derives powers corresponding to a plurality of different frequencies, respectively, from a power spectrum, and calculates a difference between the respective derived powers as an outline index. As illustrated in FIG. 8, the outline index calculation code 120c obtains each of a value of power corresponding to 3,000 hertz (Hz) and a value of power corresponding to 18,000 Hz from a power spectrum (Step S21). Subsequently, the outline index calculation code 120c calculates a power difference, which is obtained by subtracting the value of power corresponding to 18,000 Hz from the value of power corresponding to 3,000 Hz, as an outline index S1 (Step S22). As illustrated in FIG. 8, a value of the outline index S1 is prone to increase as blood viscosity obtained as blood flow data increases. The blood viscosity estimation code 120f described below takes a tendency illustrated in FIG. 8 into account, and may determine blood viscosity based on a magnitude of a value of the outline index S1. In FIG. 8, the values of powers corresponding to 3,000 Hz and 18,000 Hz are used from the power spectrum, which is merely an example. It is possible to use power corresponding to an arbitrary frequency when calculation of a power difference may be ensured. In FIG. 8, a description has been given of an example in which the outline index calculation code 120c calculates an outline index from a difference between respective powers of the power spectrum corresponding to two different frequencies, which is merely an example. For example, the outline index calculation code 120c may calculate an outline index from a difference between respective powers of the power spectrum corresponding to three different frequencies. For example, the outline index calculation code 120c may calculate arithmetic mean values of powers corresponding to a low frequency region, a middle frequency region, and a high frequency region of the power spectrum, respectively, and determine an outline index based on a difference among the three calculated arithmetic mean values. The outline index is more rarely affected by noise at the time of measurement when the arithmetic mean values are used. Further, the outline index may further represent nonlinearity based on a difference in three bands.

Using FIG. 9, a description will be given of a procedure in which the outline index calculation code 120c calculates slopes of a power spectrum corresponding to a plurality of different frequencies, respectively, as outline indices. As illustrated in FIG. 9, the outline index calculation code 120c calculates each of slope 1 between 3,000 Hz and 7,000 Hz of the power spectrum, and slope 2 between 7,000 Hz and 18,000 Hz of the power spectrum (Step S31). Subsequently, the outline index calculation code 120c calculates a ratio of slope 1 to slope 2 as an outline index S2 (Step S32). As illustrated in FIG. 9, a value of the outline index S2 is prone to increase as blood viscosity acquired as blood flow data increases. The blood viscosity estimation code 120f described below takes a tendency illustrated in FIG. 9 into account, and may determine blood viscosity based on a magnitude of a value of the outline index S2. In FIG. 9, slopes are calculated using powers at respective frequencies of 3,000 Hz, 7,000 Hz, and 18,000 Hz of the power spectrum, which is merely an example. It is possible to use power corresponding to an arbitrary frequency. As described above, an “average slope” calculated from data prior and subsequent to a frequency at which a slope is obtained (for example, 3,000 Hz, 7,000 Hz, and 18,000 Hz illustrated in FIG. 9) may be used as a slope of a tangent of the power spectrum corresponding to each of a plurality of different frequencies. The outline index is more rarely affected by noise at the time of measurement when the average slope is used.

The outline index calculation code 120c extracts an outline index corresponding to a peak of a blood flow rate calculated by the blood flow rate calculation code 120d described below from among outline indices calculated with respect to respective power spectrums. This outline index is used as target data in a process of determining blood viscosity. The peak of the blood flow rate may correspond to a maximum value of the blood flow rate in a predetermined measurement time (for example, three seconds) of the blood flow data, or a maximum value of the blood flow rate in one predetermined beat. In addition, the outline index calculation code 120c may calculate an outline index only for a power spectrum corresponding to the peak of the blood flow rate rather than calculating outline indices for all power spectrums. In addition, in order to reduce noise of the power spectrum, the outline index calculation code 120c may specify peaks of blood flow rates of a plurality of beats, average a plurality of power spectrums corresponding to the respective specified peaks, and calculate an outline index from an average power spectrum. A focus is on the peak of the blood flow rate in view of reducing a viscosity estimation error of blood due to a difference in blood flow rate as much as possible, and in view of the fact that a power spectrum corresponding to a peak time of the blood flow rate is more easily affected by blood viscosity. A minimum value of the blood flow rate in a predetermined measurement time of the blood flow data, or a minimum value of the blood flow rate in one predetermined beat may be employed as the peak of the blood flow rate.

The blood flow rate calculation code 120d provides a function for executing a process of calculating a blood flow rate based on blood flow data and a power spectrum. For example, when the blood flow data is denoted by I(t), a square mean value of I(t) is denoted by {I²}, and the power spectrum is denoted by P(f), the blood flow rate calculation code 120d calculates the blood flow rate F (a function that specifies the blood flow rate) using the following formula (1).

$$F = \frac{\int fP ( f ) df}{\int {I}^{2} dt} \qquad ( 1 )$$

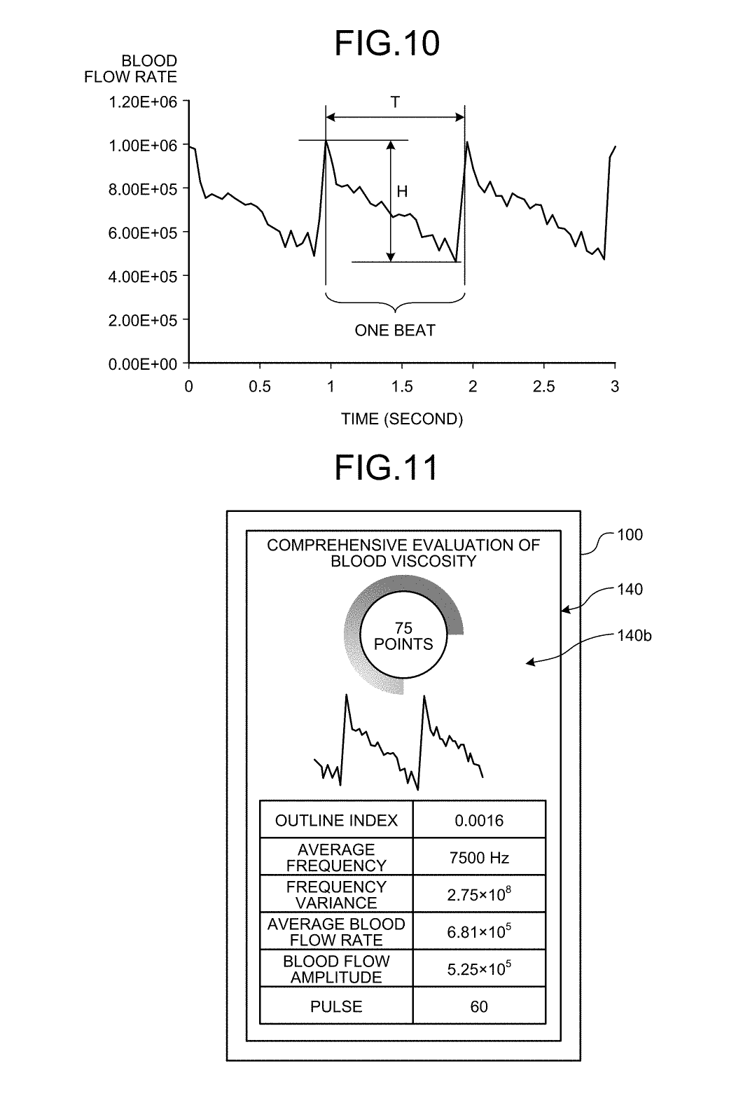

The blood flow-related information calculation code 120e provides a function for executing a process of calculating each of blood flow amplitude, an average blood flow rate, and a pulse as information related to blood. FIG. 10 is a diagram illustrating an example of a waveform representing a relation between a blood flow rate and time. A function corresponding to the waveform illustrated in FIG. 10 is calculated by the blood flow rate calculation code 120d. The blood flow-related information calculation code 120e calculates a blood flow amplitude H by extracting a difference between a maximum value and a minimum value of the blood flow rate during one beat from the waveform illustrated in FIG. 10. The blood flow-related information calculation code 120e calculates a pulse by extracting a time T corresponding to one beat from the waveform illustrated in FIG. 10.

When a frequency is denoted by f, and a power spectrum is denoted by P(f), the blood flow-related information calculation code 120e calculates an average frequency μ (a function that specifies an average frequency) using the following formula (2).

$$μ = \frac{\int fP ( f ) df}{\int P ( f ) df} \qquad ( 2 )$$

When a frequency is denoted by f, a power spectrum is denoted by P(f), and an average frequency is denoted by μ, the blood flow-related information calculation code 120e calculates a frequency variance V (a function that specifies a frequency variance) using the following formula (3).

$$V = \frac{\int {( f - μ )}^{2} P ( f ) df}{\int P ( f ) df} \qquad ( 3 )$$

The blood viscosity estimation code 120f provides a function for executing a process of determining measured blood viscosity based on an outline index corresponding to a peak of a blood flow rate. For example, the blood viscosity estimation code 120f evaluates blood viscosity based on scores of 0 to 100 by comparing the blood viscosity evaluation data 120g with an outline index extracted as target data in a process of determining blood viscosity by the outline index calculation code 120c. For example, a measurement history (outline index) of an individual user whose blood viscosity is measured, and a reference value (outline index) corresponding to the blood viscosity are accumulated as the blood viscosity evaluation data 120g. A score is calculated based on a predetermined rule in which the lower the blood viscosity, the higher the score becomes by comparing the blood viscosity evaluation data 120g, the outline index selected as the target data in the process of determining the blood viscosity, and the outline index and the reference value of the individual user. The blood viscosity estimation code 120f outputs an evaluation result of the blood viscosity to the display 140. FIG. 11 is a diagram illustrating a display example of the evaluation result of the blood viscosity. As illustrated in FIG. 11, an image 140b of an evaluation result including a score (for example, 75 points), an outline index (for example, 0.0016), an average frequency (for example, 7,500 Hz), a frequency variance (for example, 2.75×10⁸), an average blood flow rate (for example, 6.81×10⁵), a blood flow amplitude (for example, 5.25×10⁵), and a pulse (for example, 60) is displayed as a comprehensive evaluation of blood viscosity on the display 140. The image 140b of the evaluation result illustrated in FIG. 11 indicates the evaluation result of the blood viscosity as a score. However, for example, the evaluation result may be displayed as ranking in alphabetical order in which rank A ranks first, or displayed as a word that indicates a state of blood such as smooth or muddy. The image 140b of the evaluation result illustrated in FIG. 11 is an example of display. Only a score may be displayed, and advice for reducing blood viscosity may be further displayed.

The processor 130 includes hardware resources such as a central processing unit (CPU) 130a corresponding to an arithmetic unit, and a memory 130b corresponding to a storage unit, and implements various processes by executing a code stored in the storage 120 using these hardware resources. Specifically, the processor 130 reads a code corresponding to a process to be executed among various codes stored in the storage 120, and loads the code in the memory 130b. The processor 130 allows the CPU 130a to execute a command included in the code loaded in the memory 130b. The processor 130 reads and writes data to the memory 130b and the storage 120, and displays data on the display 140 based on a result of executing the command by the CPU 130a. An arithmetic processing unit may include, but is not limited to a System-on-a Chip (SoC), a Micro Control Unit (MCU), a Field-Programmable Gate Array (FPGA), a coprocessor, and the like.

For example, the processor 130 implements a process of determining a pressure on the measurement unit 110 at the time of measuring blood flow data by executing the pressure determination code 120a. For example, the processor 130 implements a process of calculating a power spectrum of the blood flow data by executing the power spectrum calculation code 120b. For example, the processor 130 implements a process of calculating an outline index from the power spectrum by executing the outline index calculation code 120c. For example, the processor 130 implements a process of calculating a blood flow rate based on the blood flow data and the power spectrum by executing the blood flow rate calculation code 120d. For example, the processor 130 implements a process of calculating each of a blood flow amplitude, an average blood flow rate, and a pulse as information related to blood by executing the blood flow-related information calculation code 120e. For example, the processor 130 implements a process of determining measured blood viscosity based on an outline index corresponding to a peak of the blood flow rate.

The display 140 includes a display device such as a liquid crystal display (LCD), an organic electro-luminescence display (OELD), or an inorganic electro-luminescence display (IELD). The display 140 displays a character, an image, a symbol, a figure, and the like. In the present embodiment, for example, the display 140 displays the image 140b of the evaluation result of the blood viscosity (see FIG. 11). For example, the display 140 displays the standby screen 140a (see FIG. 2).

The measurement unit 110 and the display 140 may include a touchscreen. When the display 140 includes the touchscreen, for example, a display and the touchscreen may be disposed to overlap each other, disposed side by side, or disposed to be separated from each other. When the display and the touchscreen are disposed to overlap each other, for example, one or a plurality of sides of the display may not be arranged along a side of the touchscreen. The touchscreen detects a contact of a finger, a pen, a stylus pen, or the like to the touchscreen. The touchscreen may detect positions on the touchscreen with which a plurality of fingers, pens, stylus pens, or the like. (hereinafter simply referred to as “fingers”) come into contact. The touchscreen notifies the processor 130 of a contact of a finger to the touchscreen together with a position on the touchscreen of a contact place. In the present embodiment, when the touchscreen is mounted on the measurement unit 110, the measurement unit 110 detects a contact of the finger F1 of the user to the measurement unit 110, and notifies the contact to the processor 130.

An arbitrary scheme such as a capacitive sensing method, a resistive membrane system, a surface acoustic wave scheme (or an ultrasonic scheme), an infrared ray system, an electromagnetic induction scheme, and a load detection scheme. may be employed as a detection scheme of the touchscreen included in the display 140.

The processor 130 may determine a type of gesture based on at least one of a contact detected by the touchscreen, a position at which the contact is detected, a change of the position at which the contact is detected, an interval at which the contact is detected, and the number of detected contacts. The gesture refers to an operation performed on the touchscreen using the finger. Examples of the gesture determined by the processor 130 through the touchscreen include, but are not limited thereto a touch, a long touch, a release, a swipe, a tap, a double tap, a long tap, a drag, a flick, pinch-in, and pinch-out.

In addition to above-described respective function units, the electronic device 100 may include a communication unit, an illuminance sensor, a proximity sensor, an acceleration sensor, a microphone, a speaker, a connector, and the like. The electronic device 100 is mounted with a function unit naturally used to maintain a function of the electronic device 100 such as a battery. When the electronic device 100 includes the illuminance sensor or the proximity sensor, an arrangement of the finger F1 of the user on the measurement unit 110 may be detected using the illuminance sensor or the proximity sensor.

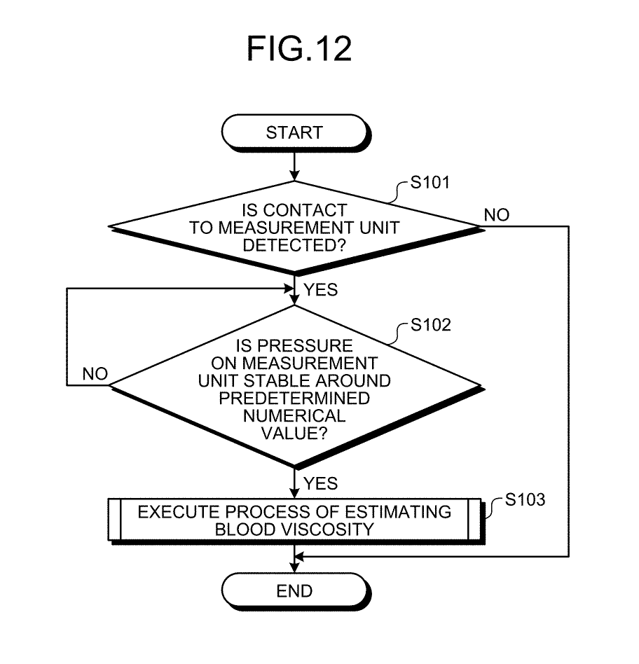

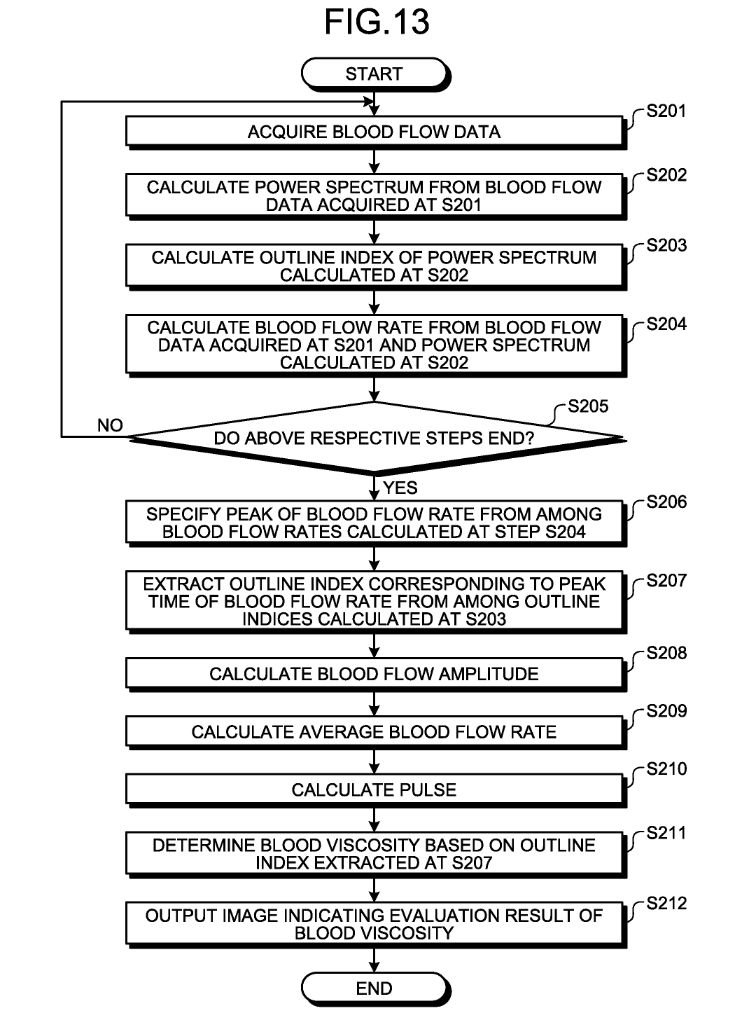

A description will be given of a flow of a process by the electronic device 100 according to some embodiments with reference to FIG. 12 and FIG. 13. FIG. 12 is a flowchart illustrating an overall flow of the process by the electronic device 100 according to embodiments. FIG. 13 is a flowchart illustrating a flow of a process of determining blood viscosity by the electronic device 100 according to some embodiments. The processes illustrated in FIG. 12 and FIG. 13 are implemented when the processor 130 executes various codes stored in the storage 120.

A description will be given of the overall flow of the process by the electronic device 100 according to embodiments using FIG. 12. As illustrated in FIG. 12, the electronic device 100 determines whether a contact to the measurement unit 110 has been detected (Step S101). That is, the electronic device 100 determines whether the finger F1 of the user has been disposed on the surface of the measurement unit 110.

When the contact to the measurement unit 110 has been detected as a result of determination (Yes at Step S101), the electronic device 100 determines whether a pressure on the measurement unit 110 is stable in a predetermined numerical value range (Step S102). For example, when the pressure on the measurement unit 110 is in a range of 2 N±0.1 N during a predetermined determination time, the electronic device 100 determines that the pressure is stable around 2 N.

When it is determined the pressure on the measurement unit 110 is unstable in the predetermined numerical value range as a result of determination (No at Step S102), the electronic device 100 repeats determination of Step S102. In contrast, when it is determined the pressure on the measurement unit 110 is stable in the predetermined numerical value range as a result of determination (Yes at Step S102), the electronic device 100 executes a process of determining blood viscosity (Step S103), and ends the process illustrated in FIG. 12.

When the contact to the measurement unit 110 is not detected as a result of determination at the above Step S101 (No at Step S101), the electronic device 100 ends the process illustrated in FIG. 12.

When a time at which the pressure on the measurement unit 110 is unstable in the predetermined numerical value range continues for a predetermined time as a result of determination at the above Step S102, the electronic device 100 may end the process illustrated in FIG. 12 by setting a timeout of the determination at Step S102.

A description will be given of the flow of the process of determining the blood viscosity by the electronic device 100 according to embodiments using FIG. 13. As illustrated in FIG. 13, the electronic device 100 acquires blood flow data acquired by the blood flow sensor 110a (Step S201).

Subsequently, the electronic device 100 calculates a power spectrum of the blood flow data from the blood flow data acquired at Step S201 (Step S202). Specifically, the electronic device 100 samples blood flow data during 0.04 second from the blood flow data acquired by the blood flow sensor 110a. Subsequently, the electronic device 100 calculates a power spectrum of the blood flow data by Fourier-transforming the sampled blood flow data. Subsequently, the electronic device 100 smoothens the calculated power spectrum.

Subsequently, the electronic device 100 calculates an outline index of the power spectrum calculated at Step S202 (Step S203). Specifically, the electronic device 100 calculates slopes at a plurality of different frequencies, a ratio of these slopes, a difference between powers at a plurality of different frequencies, and a ratio of these powers in a waveform of the power spectrum.

Subsequently, the electronic device 100 calculates a blood flow rate from the blood flow data acquired at Step S201 and the power spectrum calculated at Step S202 (Step S204). Specifically, when the blood flow data is denoted by I(t), and the power spectrum is denoted by P(f), the electronic device 100 calculates the blood flow rate F using the above formula (1).

Subsequently, the electronic device 100 determines whether to end processes of the above respective steps (Step S205). In more detail, the electronic device 100 determines whether to end the processes of the above Step S202 to Step S204 with regard to all blood flow data sampled in a predetermined measurement time (for example, three seconds) of the blood flow data.

When the processes of the above respective steps do not end as a result of determination (No at Step S205), the electronic device 100 returns to the above Step S201. In contrast, when the processes of the above respective steps end as a result of determination (Yes at Step S205), the electronic device 100 specifies a peak of the blood flow rate from the blood flow rate calculated at Step S204 (Step S206). The peak of the blood flow rate may correspond to a maximum value of the blood flow rate in the predetermined measurement time (for example, three seconds) of the blood flow data, or a maximum value of the blood flow rate in one predetermined beat.

Subsequently, from among outline indices calculated for respective power spectrums at Step S203, the electronic device 100 extracts an outline index corresponding to the peak of the blood flow rate from among outline indices calculated at Step S203 (Step S207).

Subsequently, the electronic device 100 calculates each of a blood flow amplitude, an average blood flow rate, and a pulse as information related to blood (Step S208 to Step S210).

Subsequently, the electronic device 100 determines blood viscosity based on the outline index extracted at Step S207 (Step S211). Specifically, the electronic device 100 evaluates blood viscosity based on scores of 0 to 100 by comparing the blood viscosity evaluation data 120g with an outline index extracted as target data in a process of determining blood viscosity by the outline index calculation code 120c.

Subsequently, the electronic device 100 outputs the image 140b indicating an evaluation result of the blood viscosity to the display 140 (Step S212), and ends the process illustrated in FIG. 13.

In the process illustrated in FIG. 13, a description has been given of an example in which the electronic device 100 extracts the outline index corresponding to the peak of the blood flow rate from among the outline indices calculated for the respective power spectrums at Step S203. However, embodiments are not limited thereto. For example, the electronic device 100 may calculate the outline index of the power spectrum corresponding to the peak of the blood flow rate after specifying the peak of the blood flow rate.

In the above embodiment, the electronic device 100 calculates an outline index that represents a characteristic of the waveform of the power spectrum using a numerical value from the power spectrum of the blood flow data, and determines blood viscosity based on the outline index. For this reason, the electronic device 100 may analyze blood viscosity non-invasively and in a short time.

In the above embodiment, the electronic device 100 derives powers corresponding to a plurality of different frequencies, respectively, from a power spectrum, and calculates a difference between the respective derived powers as an outline index. For this reason, the electronic device 100 may calculate the numerical value that represents the characteristic of the waveform of the power spectrum conveniently and in a short time.

In the above embodiment, the electronic device 100 calculates slopes of a power spectrum corresponding to a plurality of different frequencies as outline indices, respectively. For this reason, the electronic device 100 may calculate the numerical value that represents the characteristic of the waveform of the power spectrum conveniently and in a short time.

In the above embodiment, the electronic device 100 determines blood viscosity based on the outline index of the power spectrum corresponding to the peak of the blood flow rate. For this reason, the electronic device 100 may implement estimation of blood viscosity in view of reducing a viscosity estimation error of blood due to a difference in blood flow rate as much as possible, and in view of the fact that a power spectrum corresponding to a peak time of the blood flow rate is more easily affected by blood viscosity.

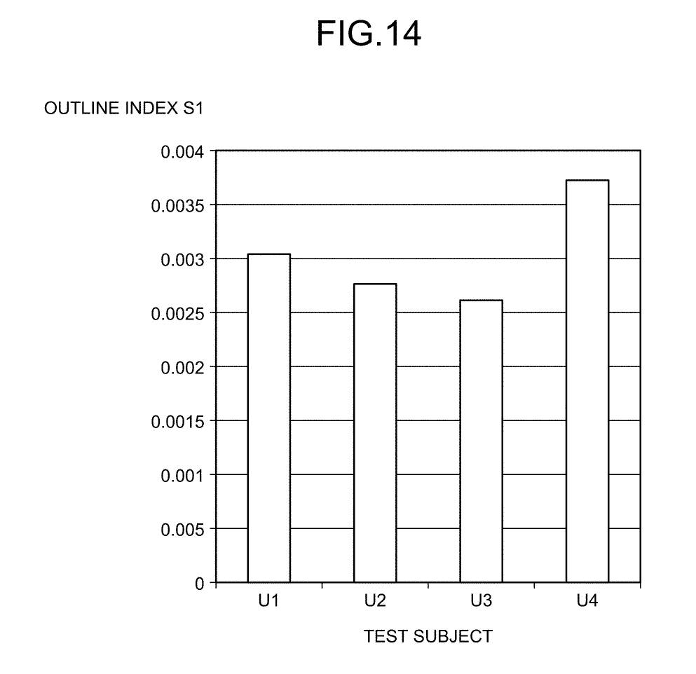

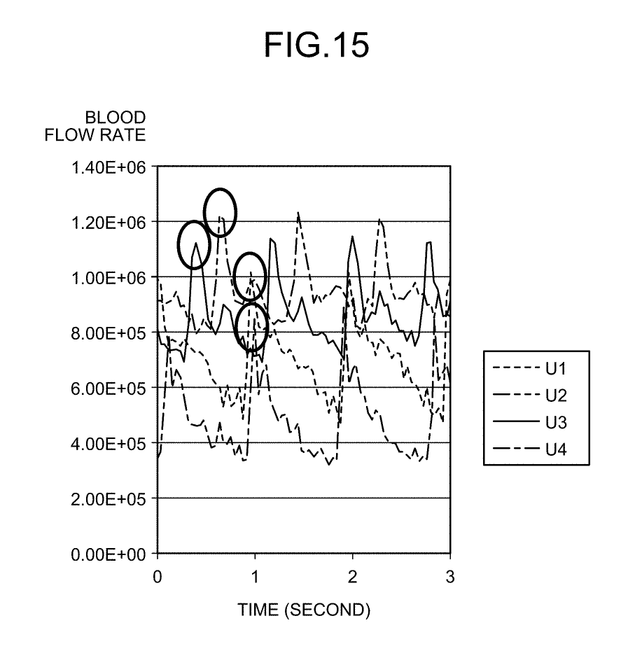

FIG. 14 is a diagram illustrating an example of outline indices corresponding to different test subjects. FIG. 15 is a diagram illustrating an example of blood flow rates corresponding to different test subjects. The outline indices illustrated in FIG. 14 and data of the blood flow rates illustrated in FIG. 15 are based on the same blood flow data. In the example illustrated in FIG. 14, a value of the outline index S1 increases in order of a test subject U3, a test subject U2, a test subject U1, and a test subject U4. In the example illustrated in FIG. 15, a blood flow rate increases in order of the test subject U4, the test subject U1, the test subject U3, and the test subject U2. In general, a magnitude of a blood flow rate and blood viscosity are in an inverse relation. That is, blood viscosity is prone to increase as a blood flow rate decreases. When an overall tendency of data illustrated in FIG. 14 and FIG. 15 is verified, the overall tendency substantially corresponds to a general tendency in which as a blood flow rate decreases, an outline index increases, and blood viscosity increases.

When the data illustrated in FIG. 14 and FIG. 15 is individually verified for each test subject, for example, the test subject U4 having a smaller blood flow rate than that of the blood flow rate U1 has a lager value of the outline index S1 than that of the test object U1, which corresponds to the general tendency in which blood viscosity increases as a blood flow rate decreases. Meanwhile, when data of the test subject U2 is compared with data of the test subject U3, the test subject U3 having a smaller blood flow rate than that of the test subject U2 has a smaller value of the outline index S1 than that of the test subject U2, which does not correspond to the general tendency in which blood viscosity increases as a blood flow rate decreases. In this way, a case in which the general tendency of the blood flow rate and blood viscosity is not applied may be considered depending on test subjects. For this reason, as in the above embodiment, it is desired to derive a tendency of blood viscosity from an outline index of a power spectrum of blood flow data, and derive a final estimation result with regard to blood viscosity (or a blood state) by synthetically considering information related to a blood flow such as a blood flow rate, a blood flow amplitude, an average blood flow rate, and a pulse. For example, in the examples illustrated in FIG. 14 and FIG. 15, a final evaluation related to blood viscosity may be derived in consideration of another piece of blood flow-related information other than an outline index. For example, when blood viscosity of the test subject U3 is determined, and when a blood flow rate and amplitude of the test subject U3 exceed relative reference values, blood viscosity may be evaluated as a low value.

In the above embodiment, a description has been given of various processing functions implemented by the electronic device 100 as an example of an electronic device according to accompanying claims. For example, the various processing functions implemented by the electronic device 100 described in the above embodiment may be mounted in a mobile device such as a smartphone and a mobile phone, and a wearable device such as a smartwatch, an activity tracker, and smart glasses.

Characteristic embodiments have been described to fully and clearly disclose a technology related to accompanying claims. However, accompanying claims should not be limited to the above embodiments, and should be embodied by all modifications and alternative configurations that may be created by those skilled in the art within a range of basic matters shown in this specification.

### REFERENCE SIGNS LIST

100 ELECTRONIC DEVICE
110 MEASUREMENT UNIT
110a BLOOD FLOW SENSOR
110b PRESSURE SENSOR
120 STORAGE
120a PRESSURE DETERMINATION CODE
120b POWER SPECTRUM CALCULATION CODE
120c OUTLINE INDEX CALCULATION CODE
120d BLOOD FLOW RATE CALCULATION CODE
120e BLOOD FLOW-RELATED INFORMATION CALCULATION CODE
120f BLOOD VISCOSITY ESTIMATION CODE
120g BLOOD VISCOSITY EVALUATION DATA
130 PROCESSOR
130a CPU
130b MEMORY

1. An electronic device comprising:
a blood flow data acquisition unit configured to acquire information related to blood flowing inside a living body as blood flow data based on Doppler shift;
a power spectrum calculator configured to calculate a power spectrum of the blood flow data based on the blood flow data; and
an outline index calculator configured to calculate an outline index from the power spectrum.

2. The electronic device according to claim 1, wherein the outline index calculator is configured to calculate each of slopes of the power spectrum corresponding to a plurality of different frequencies, respectively, as the outline index.

3. The electronic device according to claim 1, wherein the outline index calculator is configured to derive each of powers corresponding to a plurality of different frequencies, respectively, from the power spectrum, and calculate a difference between the respective derived powers as the outline index.

4. The electronic device according to claim 1, further comprising:
a blood flow rate calculator configured to calculate a blood flow rate based on the blood flow data and the power spectrum,
wherein the outline index calculator is configured to calculate the outline index based on the power spectrum corresponding to an extreme value of the blood flow rate.

5. The electronic device according to claim 1, further comprising:
an estimator configured to determine viscosity of the blood based on the outline index,
wherein the estimator is configured to display a determination result of the viscosity of the blood on a display.

6. The electronic device according to claim 4, further comprising:
a blood flow-related information calculator configured to calculate an average frequency and a frequency variance of the power spectrum, an average value of the blood flow rate, an amplitude corresponding to the blood flow rate, and a pulse corresponding to the blood flow rate; and
an estimator configured to determine viscosity of the blood based on at least one of the outline index, the average frequency and the frequency variance of the power spectrum, the average value of the blood flow rate, the amplitude corresponding to the blood flow rate, and the pulse corresponding to the blood flow rate,
wherein the estimator is configured to display a determination result of the viscosity of the blood on a display.

7. A control method executed by an electronic device, comprising the steps of:
acquiring information related to blood flowing inside a living body as blood flow data based on Doppler shift;
calculating a power spectrum of the blood flow data based on the blood flow data; and
calculating each of slopes of the power spectrum corresponding to a plurality of different frequencies, respectively, as an outline index.
